(19) United States
(12) Patent Application Publication
(10) Pub. No.: US 2007/0287829 A1
(43) Pub. Date: Dec. 13, 2007

(54) Preparation of metallotexaphyrins

(75) Inventors: Douglas Cary, Osaka (JP); Lei Fu, Freemont, CA (US); Tarak D. Mody, Sunnyvale, CA (US)

Correspondence Address: WILSON SONSINI GOODRICH & ROSATI, 650 PAGE MILL ROAD, PALO ALTO, CA, 94304-1050, US

(73) Assignee: Pharmacyclics, Inc.

(21) Appl. No.: 10/537,379

(22) PCT Filed: Nov. 24, 2003
(86) PCT No.: PCT/US03/37888

§ 371 Date: May 3, 2007

Related U.S. Application Data
(60) Provisional application No. 60/431,157, filed on Dec. 4, 2002.
(60) Provisional application No. 60/480,600, filed on Jun. 19, 2003.

(51) Int. Cl. C07F 17/00; C07D 225/00; C07F 13/00

(52) U.S. Cl. 534/14; 534/11; 534/15; 540/465

(57) ABSTRACT

The present invention provides a process for replacing a metal cation, M¹, from a compound of Formula L

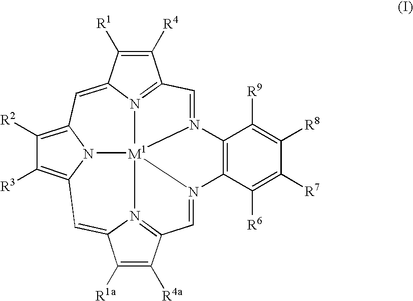

### PRIORITY INFORMATION

This application claims the benefit of priority from U.S. Provisional Application No. 60/431,157, filed Dec. 4, 2002 (Attorney Docket No. 4235.00 US) and U.S. Provisional Application No. 601480,600, filed Jun. 19, 2003 (Attorney Docket No. 4235.01 US), both of which are incorporated by reference herein their entirety.

### FIELD OF INVENTION

The present invention relates to a transmetallation process for the synthesis of texaphyrin compounds of Formula II.

### BACKGROUND OF INVENTION

Texaphyrins are synthetic porphyrin-like ligands which complex large metal cations, including the trivalent cations of the lanthanide series. Texaphyrins have the potential of treating diseases like cancer, cardiovascular diseases, infectious diseases, and the like. One derivative, motexafin gadolinium (Xcytrin®, MGd), has been reported to localize to tumors and to enhance radiation response in animal tumor models. This drug is currently in late stage clinical development as an adjuvant for radiation therapy of brain metastases.

Given the utility of texaphyrins one needs to synthesize a diverse array of these compounds to study their utility. Texaphyrin synthesis had been reported, for example, in U.S. Pat. Nos. 5,162,509, 5,530,122 and 5,569,759. The processes described are cumbersome, have limitations and have permitted synthesis of texaphyrins with limited number of metal cations. There is thus a need of new processes that can permit synthesis of a wider array of texaphyrins.

### SUMMARY OF THE INVENTION

The present invention provides a process of synthesizing texaphyrins of Formula II: 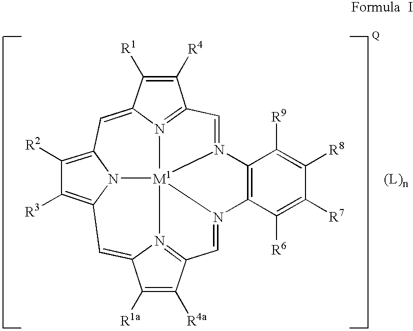
wherein:
M1 represents a metal cation selected from Ca⁺² and Mg⁺²;
Q represents an integer of from about −5 to about +5;
L represents a charge balancing species;
“n” represents an integer of from 0 to +5;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO2—R³¹, and the moiety X—Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;
X is a covalent bond or a linker;
Y is a catalytic group, a chemotherapeutic agent or a site-directing group; and
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl;
to form a compound of Formula II with a metal cation M² 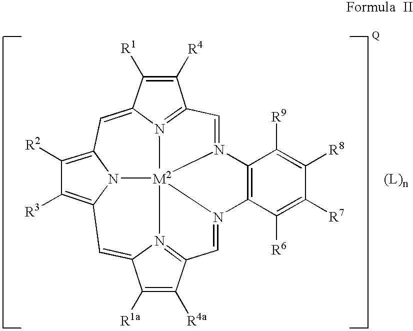
   wherein:
M² represents a metal cation selected from TI⁺³, Ti⁺³, In⁺³, Cr⁺², Mn⁺² , Fe⁺², Gd⁺³, Co⁺³, Ni⁺², Zn⁺², Yb⁺², Cd⁺², Nd⁺³, Sm⁺³ , Eu⁺³, Tb⁺³, Dy⁺³, Y⁺³, Fe⁺³, Ga⁺³, Bi⁺³, Lu⁺³, Tc⁺², Tc⁺³, Tc⁺⁴, U⁺³, Np⁺³, Pu⁺³, Am⁺³, Cm⁺³ and Cf⁺³;
Q, L, “n” R¹, R^{1a}, R², R³, R⁴, R^{4a}, R⁷, and R⁸ are as indicated above;
said process comprising treating, in a suitable medium, a compound of Formula I with a compound of formula A
   M²-Z₍₂₋₄₎ formula A
   wherein M² is a s defined above and Z represents OAc, PO₄, NO₃, OTFA, AcAc, Br, I or Cl, optionally in the presence of a base and at a temperature of from about 25° C. to about 100° C., to form a compound of Formula II.

### DETAILED DESCRIPTION OF THE INVENTION

The present invention provides a process of synthesizing texaphyrins of Formula II: 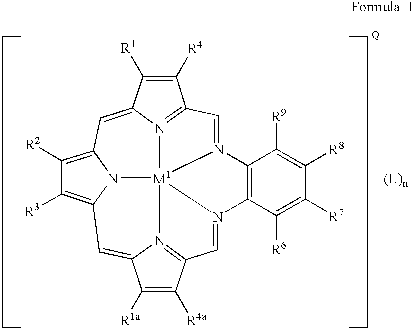
wherein:
M¹ represents a metal cation selected from Ca⁺² and Mg⁺²;
Q represents an integer of from about −5 to about +5;
L represents a charge balancing species;
“n” represents an integer of from 0 to +5;
R¹, R^{1a}, R², R³, R⁴, R^{4a}, R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO2—R³¹, and the moiety X—Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;
X is a covalent bond or a linker;
Y is a catalytic group, a chemotherapeutic agent or a site-directing group; and
R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl;
to form a compound of Formula II with a metal cation M² 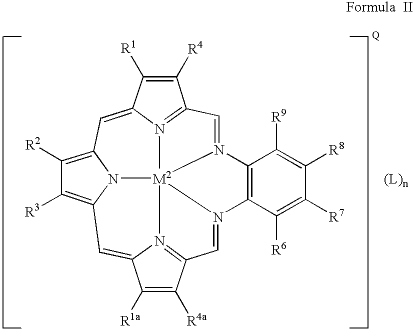
   wherein:
M² represents a metal cation selected from TI⁺³, Ti⁺³, In⁺³, Cr⁺², Mn⁺², Fe⁺², Gd⁺³, Co⁺³, Ni⁺², Zn⁺², Yb⁺², Cd⁺², Nd⁺³, Sm⁺³ , Eu⁺³, Tb⁺³, Dy⁺³, Y⁺³, Fe⁺³, Ga⁺³, Bi⁺³, Lu⁺³, Tc⁺², Tc⁺³, Tc⁺⁴, U⁺³, Np⁺³, Pu⁺³, Am⁺³, Cm⁺³ and Cf⁺³,
Q, L, “n” R¹, R^{1a}, R², R³, R⁴, R^{4a}, R⁷, and R⁸ are as indicated above;
said process comprising treating, in a suitable medium, a compound of Formula I with a compound of formula A
   M²-Z₍₂₋₄₎ formula A
wherein M² is as defined above and Z represents OAc, PO₄, NO₃, OTFA, AcAc, Br, I or Cl, optionally in the presence of a base and at a temperature of from about 25° C. to about 100° C., to formula II.

A preferred embodiment provides a process wherein M² represents TI⁺³, In⁺³, Mn⁺², Fe⁺², Gd⁺³, Co⁺³, Dy⁺³, Fe⁺³, Bi⁺³, Lu⁺³, Y⁺³, Tc⁺², Tc⁺³, or Tc⁺⁴; and L is selected from OAc, PO₄, NO₃, OTFA, AcAc, Br, I and Cl.

Another preferred embodiment provides a process wherein M² represents TI⁺³, Mn⁺², Gd⁺³, Co⁺³, In⁺³, Bi⁺³, Dy⁺³, Y^{30 3}, or Lu⁺³, and the suitable medium is selected from EtOH, MeOH, DMF, CH₂Cl₂, CHCl₃, THF, IPA, pyridine, 2,6-lutidine, CH₃CN, Et₃N, DMSO, acetyl acetone and mixtures thereof. A further preferred embodiment provides a process wherein the suitable medium is selected from EtOH, MeOH, CH₃CN, or mixtures thereof.

Provided in another embodiment of the present invention is process wherein the temperature ranges from about 40° C. to about 80° C., with a temperature range of from about 60° C. to about 70° C. being more preferred. Another preferred embodiment provides a process wherein Z in formula A is selected from Cl and OAc.

The present invention in another one of its preferred embodiments provides a process wherein the compound of formula A are selected from InCl₃, In(OAc)₃, TI(OAc)₃, Gd(OAc)₃, Lu(OAc)₃, and Mn(OAc)₂, and the metal M² is selected from TI⁺³, Mn⁺², Gd⁺³, In⁺³ and Lu⁺³.

Another aspect of the present invention provides a process wherein the optional base is selected from sodium acetate, sodium citrate, pyridine, 2,6-lutidine, triethyl amine, and sodium phosphate. A further preferred embodiment of this aspect of the invention provides a process wherein the compound of Formula I is treated with a compound of formula A in the presence of a base selected from sodium acetate, sodium citrate, triethylamine, and 2,6-lutidine, with sodium acetate, triethylamine, and 2,6-lutidine being more preferred.

Experimental Details

General Procedure

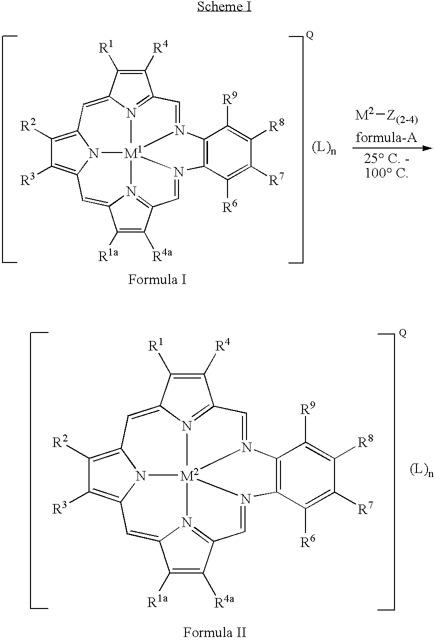

A mixture of a compound of formula -A (M²-Z³; 1 eq.) in a suitable medium (ethanol) was agitated in a reaction vessel equipped with a stirrer bar and a reflux condensor. To the agitating reaction mixture then was added a compound of Formula I (e.g., M¹=Ca; 1 eq). The resulting reaction mixture then was agitated for about 60 minutes at a temperature of from about 25° C. to about 100° C. The reaction mixture then was cooled to ambient temperature. The desired product, compound of Formula II, can be isolated by techniques, such as chromatography, known to one skilled in the art.

### EXAMPLES:

### Example 1

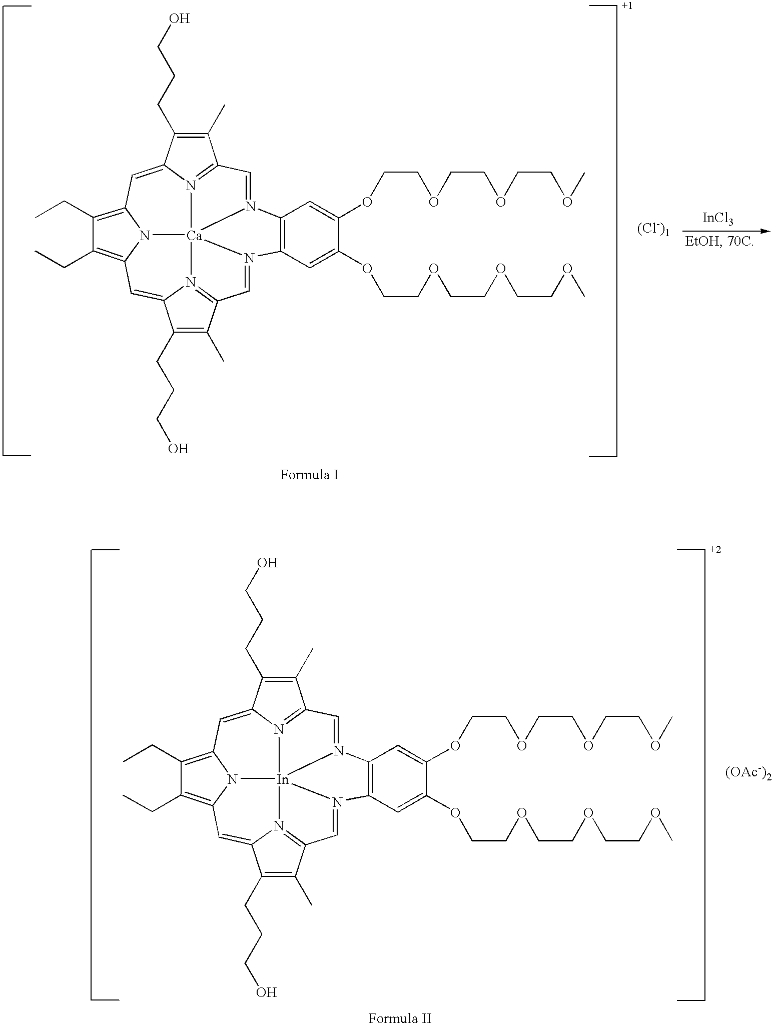 Procedure 1

A mixture of a compound of formula -A (InCl₃; 0.1054 mM) and Ethanol (˜50 mL) was agitated at ambient temperature in a reaction vessel equipped with a reflux condenser and a stirrer bar. The reaction vessel was maintained under an atmosphere of nitrogen. A compound of Formula I (M¹=Ca; 0.1054 mM) then was added to the agitating reaction mixture followed by about 10 mL ethanol. The resulting mixture then was agitated at a temperature of about 70° C. for about 60 minutes. The reaction mixture then was cooled to ambient temperature. The compound of Formula II was then isolated by subjecting the reaction mixture to reversed phase chromatography as described below.

Procedure 2

A mixture of a compound of formula -A (InCl₃; 0.1054 mM), Ethanol (˜50 mL), sodium acetate (3.0477 mM) and 1 mL of 0.05 mM HCl was agitated at ambient temperature in a reaction vessel equipped with a reflux condensor and a stirrer bar. The reaction vessel was maintained under an atmosphere of nitrogen. A compound of Formula I (M¹=Ca; 0.1054 mM) then was added to the agitating reaction mixture followed by about 10 mL ethanol. The resulting mixture then was agitated at a temperature of about 70° C. for about 60 minutes. The reaction mixture then was cooled to ambient temperature. The compound of Formula II was then isolated by subjecting the reaction mixture to reversed phase chromatography as described below.

Reversed Phase Chromatography

Two reversed-phase SepPak™ columns (tC18, 10 g) were conditioned by washing each column with 20 mL of MeOH, followed by 20 mL of 100 mM Ammonium Acetate (AA) buffer solution (pH 4.3). The above cooled reaction mixture was diluted with 100 mM AA buffer solution. To one of the prepared SepPak™ columns, the diluted reaction mixture was added, in aliquots, and allowed to flow through the column. The process was repeated until all of the reaction mixture was loaded onto the column. The In-Tex was then eluted from the column with 50 mL of a solvent mixture having a ratio of 3:2 v/v of MeOH and 100 mM AA buffer. The In-Tex eluted from the first column with the free base texaphyrin and the other impurities remaining on the column. The elute collected from the column was diluted with 100 mM AA buffer and loaded onto the second SepPak™ column. The In-Tex was retained on the column matrix and washed with Dl water to wash away the residual salts. Afterwards, the compound of Formula II (Indium Texaphyrin, M²=In) was eluted off the column with 30 mL of MeOH. The elute was collected and the solvent removed under reduced pressure.

Compound Characterization:

Characterization data for examples which were prepared by using Procedures 1 and 2 are as follows:

### Example 1

M²=In⁺³
Analyzed by HPLC, UV-vis, and LC/MS
UV-vis analysis: Amax: 468 nm and 730 nm
MS analysis: found 1046.3 (theoretical 1046.4 for C₅₀H₆₉N₅O₁₂In⁺)

### Example 2

M²=Y⁺³
Analyzed by HPLC, UV-vis, and LC/MS
UV-vis analysis: Amax: 472 nm and 732 nm
MS analysis: found 1020.4 (theoretical 1020.4 for C₅₀H₆₉N₅O₁₂Y⁺)

### Example 3

M²=Gd⁺³
Analyzed by HPLC, UV-vis, and LC/MS
UV-vis analysis: Amax: 470 nm and 740 nm
MS analysis: found 1089.3 (theoretical 1089.4 for C₅₀H₆₉N₅O₁₂Gd⁺)

### Example 4

M²=Lu⁺³
Analyzed by HPLC, UV-vis, and LC/MS
UV-vis analysis: Amax: 472 nm and 732 nm
MS analysis: found 1106.4 (theoretical 1106.4 for C₅₀H₆₉N₅O₁₂Lu⁺)

### Example 5

M²=Mn⁺³
Analyzed by HPLC, UV-vis, and LC/MS
UV-vis analysis: Amax: 460 nm and 728 nm
MS analysis: found 927.5 (theoretical 927.4 for C₄₈H₆₆N₅O₁₀Mn⁺)

### Example 6

M²=TI⁺³
Analyzed by HPLC, UV-vis, and LC/MS
UV-vis analysis: Amax: 472 nm and 738 nm
MS analysis: found 1136.3 (theoretical 1136.4 for C₅₀H₆₉N₅O₁₂TI⁺)

### Example 7

M²=Nd⁺³
Analyzed by HPLC, UV-vis, and LC/MS
UV-vis analysis: Amax: 472 nm and 744 nm
MS analysis: found 1075.4 (theoretical 1074.4 for C₅₀H₆₉N₅O₁₂Nd⁺)

### Abbreviations:

IPA: Isopropyl alcohol
AcAc: Acetoacetone
OTFA: O-trifluoro acetate (OCOCF₃)
OAc: OCOCH₃

### Definitions

As used in the present specification, the following words and phrases are generally intended to have the meanings as set forth below, except to the extent that the context in which they are used indicates otherwise.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined below. It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible.

The term “suitable medium”, as used herein is intended to represent solvents that can facilitate the exchange of metal cation M¹ for metal cation M². One skilled in the art can determine a solvent that can facilitate such an exchange through routine experimentation. One of the characteristics of a suitable medium is that it at least partially dissolves the reactants and reagents used in the present invention. Illustrative examples of a suitable solvent are EtOH, MeOH, THF, DMF, DMSO, CH₂Cl₂ and the like.

The term “base”, as used herein, is intended to represent a chemical agent that neutralizes protons and/or acidic species. Illustrative examples of a base are sodium acetate, sodium citrate, pyridine, 2,6-lutidine, triethyl amine, and sodium phosphate.

The term “acyl” refers to the groups —C(O)—H, —C(O)-(optionally substituted alkyl), —C(O)-(optionally substituted cycloalkyl), —C(O)-(optionally substituted alkenyl), —C(O)-(optionally substituted cycloalkenyl), —C(O)-(optionally substituted aryl), —C(O)-(optionally substituted heteroaryl) and —C(O)-(optionally substituted heterocyclyl).

The term “acyloxy” refers to the moiety —O-acyl, including, for example, —O—C(O)-alkyl.

The term “alkoxy” refers to the groups' —O-alkyl, —O-alkenyl, —O-cycloalkyl, —O-cycloalkenyl, and —O-alkynyl. Preferred alkoxy groups are —O-alkyl and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups —O-(substituted alkyl), —O-(substituted alkenyl), —O-(substituted cycloalkyl), —O-(substituted cycloalkenyl), —O-(substituted alkynyl) and —O-(optionally substituted alkylene)-alkoxy. One preferred substituted alkoxy group is “polyalkoxy” or —O-(substituted alkylene)-alkoxy, and includes groups such as —OCH₂CH₂OCH₃, and (or PEG) groups such as —O(CH₂CH₂O)ₓCH₃, where x is an integer of about 2-20, preferably about 2-10, and more preferably about 2-5. Another preferred substituted alkoxy group is —O-(substituted,alkyl), and includes groups such as —OCH₂(CH₂)_{y}OH, where y is an integer of about 1-10, preferably about 1-4.

The term “alkoxyalkylene” refers to the groups: -alkylene-O-alkyl, -alkylene-O-(substituted alkyl), -(substituted alkylene)-O-alkyl and -(substituted alkylene)-O-(substituted alkyl). A preferred alkoxyalkylene group is -alkylene-O-alkyl and include, by way of example, methoxymethylene (—CH₂OCH₃), methoxyethylene (—CH₂CH₂OCH₃), n-(iso-propoxy)propylene [—CH₂CH₂CH₂OCH(CH₃)₂] and the like.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having at least 1 and preferably from 1-6 sites of vinyl unsaturation. Preferred alkenyl groups include ethenyl (—CH═CH₂), 2-propen-1-yl (—CH₂CH═CH₂), isopropenyl [—C(CH₃)═CH₂], and the like.

The term “substituted alkenyl” refers to an alkenyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfonyl.

The term “alkenylene” refers to a diradical derived from the above-defined monoradical, alkenyl. This term is exemplified by groups such as ethenylene (—CH═CH—), the propenylene isomers (e.g., —CH₂CH═CH— and —C(CH₃)═CH—) and the like.

The term “substituted alkenylene” refers to a diradical derived from the above-defined monoradical, substituted alkenyl.

The term “alkyl” refers to a monoradical branched or unbranched saturated hydrocarbon chain preferably having from about 1 to 20 carbon atoms, more preferably about 1 to 10 carbon atoms, and even more preferably about 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbony-l, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, phosphono, sulfanyl, sulfinyl, and sulfonyl.

One of the preferred optional substituents for alkyl is hydroxy, exemplified by hydroxyalkyl groups, such as 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and the like; and those compounds known as polyethylene glycols, polypropylene glycols and polybutylene glycols, and the like.

The term “alkylene” refers to a diradical derived from the above-defined monoradical, alkyl. This term is exemplified by groups such as methylene (—CH₂—), ethylene (—CH₂CH₂—), the propylene isomers [e.g., —CH₂CH₂CH₂— and —CH(CH₃)CH₂—] and the like.

The term “substituted alkylene” refers to a diradical derived from the above-defined monoradical, substituted alkyl. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethylene (CH(OCH₂CH₃)CH₂), 3-oxapentylene (—CH₂CH₂O—CH₂CH₂—), N-methyl-3-azapentylene, (—CH₂CH₂N(CH₃)CH₂CH₂—), 3,6,9-trioxaundecylene (2-ethoxy-ethoxy)ethylene (—CH₂CH₂O—CH₂CH₂—OCH₂CH₂—OCH₂CH₂—), and the like.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (or propynyl, —C≡CCH₃), and the like.

The term “substituted alkynyl” refers to an alkynyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony.

The term “alkynylene” refers to a diradical derived from the above-defined monoradical, alkynyl. Preferred alkynylene groups include ethynylene (—C≡C—), propargylene (—CH₂—C≡C—) and the like.

The term “substituted alkynylene” refers to a diradical derived from the above-defined monoradical, substituted alkynyl.

The term “amino” refers to the group —NH₂.

The term “substituted amino” refers to the group —NHR or —NRR where each R is independently selected from the group: acyl, optionally substituted alkenyl, optionally substituted alkyl, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, and optionally substituted heterocyclyl. Preferred amino substituents include optionally substituted alkyl, aryl, optionally substituted alkoxycarbonyl (also referred to as a “carbamate”), optionally substituted aminocarbonyl (also referred to as a urea) and heteroaryl.

The term “apical ligand” refers to an anion that binds to the core metal of the metallotexaphyrin, e.g., with de-localized electrostatic or weak coordinate-covalent bonds. The number of apical ligands (n) is defined as an integer of 0-5. It should be noted that the apical ligands act to neutralize the charge on the metallotexaphyrin. Thus, typically n is 1 when M is a divalent cation, and n is 2 when M is a trivalent cation (because the core itself neutralizes one unit charge). However, if any of R¹, R^{1a}, R², R³, R⁴, R^{4a}, R⁶, R⁷, R⁸, and R⁹ is capable of forming an acid addition salt, for example a carboxylate or a phosphate, then n will decrease appropriately. It is also possible that the apical ligands could have two functionalities capable of forming an anion, for example a dicarboxylic acid, and such ligands are intended to be within the scope of the invention. In general, any molecule containing a carboxylic acid or phosphate may be used as an apical ligand, for example biomolecules, including lipoproteins, estradiol and amino acids, carboxylates of sugar derivatives, such as gluconic acid or glucoronic acid, cholesterol derivatives such as cholic acid and deoxycholic acid, PEG acids, organophosphates, such as methylphosphonic acid and phenylphosphonic acid, and phosphoric acid or other inorganic acids, and the like, or sulfonic acid derivatives such as methanesuffonic acid, ethanesulfonic acid, or “carboxylic acid derivatives”, which term refers to compounds of the formula R—CO₂H, in which R is optionally substituted alkyl, optionally substituted alkenyl, optionally substituted alkynyl, or optionally substituted aryl, as defined above. Preferred are gluconic and glucuronic acid, and those carboxylic acid derivatives where R is optionally substituted alkyl, for example acids of 1-20 carbon atoms, such as formic acid, acetic acid, propionic acid, butyric acid, pentanoic acid, 3,6,9-trioxodecanoic acid, 3,6-dioxoheptanoic acid, methylvaleric acid, glycolic acid, pyruvic acid, oxalic acid, malic acid, malonic acid, succinic acid, maleic acid, fumaric acid, tartaric acid, citric acid, and the like. Also preferred are those carboxylic acid derivatives where R is aryl, in particular where R is optionally substituted phenyl, for example benzoic acid, salicylic acid, 3-fluorobenzoic acid, 4-aminobenzoic acid, cinnamic acid, mandelic acid, p-toluene-sulfonic acid, and the like.

The term “aromatic” refers to a cyclic or polycyclic moiety having a conjugated unsaturated (4ₙ+2) π electron system (where n is a positive integer), sometimes referred to as a delocalized it electron system.

The term “aryl” refers to an aromatic cyclic hydrocarbon group of from 6 to 20 carbon atoms having a single ring (e.g., phenyl) or multiple condensed (fused) rings (e.g., naphthyl or anthryl). Preferred aryls include phenyl, naphthyl and the like.

The term “substituted aryl” refers to an aryl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony (except as otherwise constrained by the definition for the aryl substituent).

The term “aryloxy” refers to the group —O-aryl.

The term “substituted aryloxy” refers to the group —O-(substituted aryl).

The term “arylalkyl” refers to the moiety “-alkylene-aryl” each having the meaning as defined herein. Such arylalkyl groups are exemplified by benzyl, phenethyl, 3-naphthylpropyl and the like. Arylalkyl moieties also fall within the definition of optionally substituted alkyl, e.g., as a 2-phenyl-n-pentyl moiety.

The term “substituted arylalkyl” refers to the moiety “-(optionally substituted alkylene)- (optionally substituted aryl),” each having the meaning as defined herein, where at least one of the aryl or alkylene groups is substituted, e.g., 4-(N-methyl-pyrrolyl)pentylene.

The term “carbonyl” refers to the di-radical “—C(═O)—”, which is also written as “—C(O)—”.

The term “(optionally substituted alkoxy)carbonyl” refers to the groups: —C(O)O-substituted alkyl), —C(O)O-(optionally substituted cycloalkyl), —C(O)O-(optionally substituted alkenyl), and —C(O)O-(optionally substituted alkynyl). These moieties are also referred to as esters.

The term “(optionally substituted amino)carbonyl” refers to the group —C(O)-(optionally substituted amino). This moiety is also referred to as a primary, secondary or tertiary carboxamide.

The term “(optionally substituted alkyl)carbonyloxy” refers to the group —O—C(O)-(optionally substituted alkyl). This moiety is also referred to as a “carbonate.”

The term “(optionally substituted amino)carbonyloxy” refers to the group —O—C(O)-(optionally substituted amino). This moiety is also referred to as a “carbamate.”

The term “carboxy” or “carboxyl” refers to the moiety “—C(O)OH”, which is also illustrated as “—COOH”.

The term “cycloalkyl” refers to non-aromatic cyclic hydrocarbon groups having about 3 to 40 (preferably about 4 to 15) carbon atoms having a single ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to a cycloalkyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, optionally substituted azo, phosphono, sulfanyl, sulfinyl, and sulfony (except as otherwise constrained by the definition for the cycloalkyl substituent).

The term “cycloalkylene” refers to a diradical derived from the above-defined monoradical, cycloalkyl, and is exemplified by 1,1-cyclopropylene, 1,2-cyclobutylene, 1,4-cyclohexylene and the like.

The term “substituted cycloalkylene” refers to the diradical derived from substituted cycloalkyl as defined above.

The term “heteroaryl” refers to an aromatic cyclic hydrocarbon group having about 1 to 40 (preferably from about 3 to 15) carbon atoms and about 1 to 10 hetero atoms (preferably about 1 to 4 heteroatoms, selected from nitrogen, sulfur, phosphorus, and/or oxygen) within at least one ring. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

The term “substituted heteroaryl” refers to a heteroaryl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, optionally substituted azo, phosphonato, phosphono, sulfanyl, sulfinyl, and sulfony (except as otherwise constrained by the definition for the heteroaryl substituent).

The term “heteroaryloxy” refers to the group —O-heteroaryl. It is understood that the metals represented by M² in compounds of Formula II include all the isotopes of the respective metals. Thus, for example, when M² represents Indium (In) it is understood that all isotopes of Indium are included in the definition of M². A list of all the metal isotopes can be found in the Handbook of Chemistry and Physics, 82nd edition, 2001-2002, David R. Lide editor-in-chief and is incorporated herein by reference. Illustrative examples of the metal isotopes are 90Y⁺³, 153Gd⁺³, 111In⁺³, 115Cd⁺², 210Bi⁺³, 147Nd⁺³, 153Sm⁺³, 166Dy⁺³ and 177Lu⁺³.

The term “heteroarylene” refers to the diradical group derived from heteroaryl (including substituted heteroaryl), as defined above, and is exemplified by the groups 2,6-pyridylene, 2,4-pyridylene, 1,2-quinolinylene, 1,8-quinolinylene, 1,4-benzofuranylene, 2,5-pyridylene, 2,5-indolylene and the like.

The terms “heterocycle”, “heterocyclic” and “heterocyclyl” are interchangeable, and refer to a monoradical, saturated or unsaturated, non-aromatic cyclic hydrocarbon group having from about 3 to about 40 (preferably from about 3 to about 15) carbon atoms wherein one to about 10 carbon atoms are independently replaced hetero atoms selected from nitrogen, sulfur, phosphorus, oxygen, and selenium. In a preferred embodiment about 1 to about 4 carbon atoms are replaced by hetero atoms. Such heterocyclic groups can have a single ring or multiple condensed rings. Illustrative examples of a heterocycle are morpholino, piperidinyl, and the like.

The terms “substituted heterocycle”, “substituted heterocyclic” and “substituted heterocyclyl” refer to a heterocyclyl group in which 1 or more (up to about 5, preferably up to about 3) hydrogen atoms is replaced by a substituent independently selected from the group: ═O, ═S, acyl, acyloxy, optionally substituted alkoxy, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, halogen, optionally substituted heteroaryl, optionally substituted heteroaryloxy, optionally substituted heterocyclyl, optionally substituted heterocyclooxy, hydroxyl, nitro, optionally substituted phosphine, phosphonato, optionally substituted azo, phosphono, sulfanyl, sulfinyl, and sulfony (except as otherwise constrained by the definition for the heterocyclic substituent).

The term “heterocyclylooxy” refers to the group —O-heterocycle.

The term “heterocyclylene” refers to the diradical group formed from a heterocycle, as defined herein, and is exemplified by the groups 2,6-morpholino, 2,5-morpholino and the like.

1. A process for replacing a metal cation, M¹, from a compound of Formula I 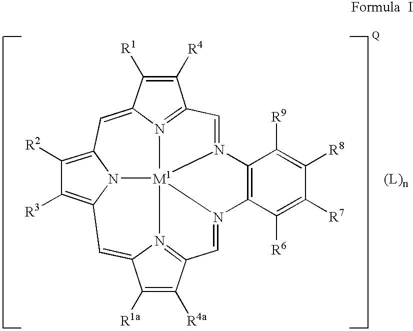
wherein:
M¹ represents a metal cation selected from Ca⁺² and Mg⁺²;
Q represents an integer of from about −5 to about +5;
L represents a charge balancing species;
“n” represents an integer of from 0 to +5;
R¹, R^{1a}, R², R³, R⁴, R^{4a}a R⁷, and R⁸ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, halogen, optionally substituted heteroaryl, optionally substituted heteroaryloxy, optionally substituted heterocyclyl, optionally substituted heterocyclooxy, hydrogen, hydroxyl, nitro, optionally substituted azo, S—R³¹, SO—R³¹, SO2—R³¹, and the moiety X—Y;
R⁶ and R⁹ are independently selected from acyl, acyloxy, optionally substituted alkenyl, optionally substituted alkoxy, optionally substituted alkyl, optionally substituted alkynyl, optionally substituted amino, optionally substituted aryl, optionally substituted aryloxy, carboxyl, (optionally substituted alkoxy)carbonyl, (optionally substituted amino)carbonyl, (optionally substituted alkoxy)carbonyloxy, (optionally substituted amino)carbonyloxy, cyano, optionally substituted cycloalkyl, optionally substituted cycloalkenyl, fluoro, chloro, bromo, optionally substituted heteroaryl, optionally substituted heteroaryloxy, optionally substituted heterocyclyl, optionally substituted heterocyclooxy, hydrogen, hydroxyl, nitro, optionally substituted azo, sulfanyl, sulfinyl, sulfonyl, and the moiety X—Y;
X is a covalent bond or a linker;
Y is a catalytic group, a chemotherapeutic agent or a site-directing group; and R³¹ represents acyl, optionally substituted alkenyl, optionally substituted alky, optionally substituted alkoxy, optionally substituted alkoxycarbonyl, optionally substituted alkynyl, optionally substituted aminocarbonyl, optionally substituted aryl, carboxy, optionally substituted cycloalkyl, optionally substituted heteroaryl, or optionally substituted heterocyclyl;
to form a compound of Formula II with a metal cation M² 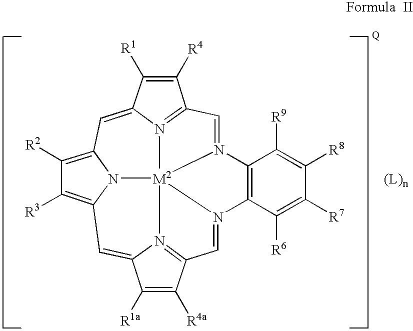
wherein: M² represents a metal cation selected from TI⁺³, Ti⁺³, In⁺³, Cr⁺², Mn⁺², Fe⁺², Gd⁺³, Co⁺³, Ni⁺², Zn⁺², Yb⁺², Cd⁺², Nd⁺³, Sm⁺³, Eu⁺³, Tb⁺³, Dy⁺³, Y⁺³, Fe⁺³, Ga⁺³, Bi⁺³, Lu⁺³, Tc⁺² Tc⁺³, Tc⁺⁴, U⁺³, Np⁺³, Pu⁺³, Am⁺³, Cm⁺³ and Cf⁺³,
Q, L, “n”, R¹, R^{1a}, R², R³, R⁴, R^{4a}, R⁷, and R⁸are as indicated above;
said process comprising treating, in a suitable medium, a compound of Formula I with a compound of formula A
M²-Z₍₂₋₄₎ formula A
wherein M² is as defined above and Z represents OAc, PO₄, NO₃, OTFA, AcAc, Br, I or Cl, optionally in the presence of a base and at a temperature of from about 25° C. to about 100° C., to form a compound of Formula II.

2. A process of claim 1 wherein M² represents TI⁺³, In⁺³, Mn⁺², Fe⁺², Gd⁺³, Co⁺³ , Dy⁺³, Y⁺³, Fe⁺³, Bi⁺³, Lu⁺³, Y⁺³, Tc⁺², Tc⁺³ or Tc⁺⁴; and
L is selected from OAc, PO₄, NO₃, OTFA, AcAc, Br, I and Cl.

3. A process of claim 2 wherein M² represents TI⁺³, Mn⁺², Gd⁺³, Co⁺³, In⁺³, Bi⁺³, Dy⁺³, Y⁺³ or Lu⁺³.

4. A process of claim 3 wherein the suitable medium is selected from EtOH, MeOH, DMF, CH₂Cl₂, CHCl₃, THF, IPA, pyridine, 2,6-lutidine, CH₃CN, Et₃N, DMSO, acetyl acetone and mixtures thereof.

5. A process of claim 4 wherein the suitable medium is selected from EtOH, MeOH, CH₃CN, or mixtures thereof.

6. A process of claim 5 wherein the temperature ranges from about 40° C. to about 80° C.

7. A process of claim 6 wherein the temperature ranges from about 60° C. to about 70° C.

8. A process of claim 7 wherein Z in formula A is selected from Cl and OAc.

9. A process of claim 8 wherein the compound of formula A are selected from InCl₃, In(OAc)₃, TI(OAc)₃, Gd(OAc)₃, Lu(OAc)₃, and Mn(OAc)₂.

10. A process of claim 9 wherein the metal M² is selected from TI⁺³, Mn⁺², Gd⁺³, In⁺³ and Lu⁺³.

11. A process of claim 9 wherein the optional base is selected from sodium acetate, sodium citrate, pyridine, 2,6-lutidine, triethyl amine, and sodium phosphate.

12. A process of claim 11 wherein the compound of Formula I is treated with a compound of formula A in the presence of a base selected from sodium acetate, sodium citrate, triethylamine, and 2,6-lutidine.

13. A process of claim 12 wherein the base is selected from sodium acetate, triethylamine, and 2,6-lutidine.
